(19) United States
(12) Patent Application Publication
(10) Pub. No.: US 2005/0209606 A1
(43) Pub. Date: Sep. 22, 2005

(54) Alginate viscoelastic composition, method of use and package

(76) Inventors: Dharmendra Jani, Fairport, NY (US); Joseph C. Salamone, Fairport, NY (US)

Correspondence Address: Bausch & Lomb Incorporated, One Bausch & Lomb Place, Rochester, NY, 14604-2701, US

(21) Appl. No.: 11/064,593

(22) Filed: Feb. 24, 2005

Related U.S. Application Data
(60) Provisional application No. 60/547,855, filed on Feb. 26, 2004.

(51) Int. Cl. A61F 9/013; C08B 37/04

(52) U.S. Cl. 606/107; 128/898; 206/364; 623/905; 536/3

(57) ABSTRACT

The present invention is a novel viscoelastic composition or material that includes a suitable aqueous carrier and alginate. The novel viscoelastic composition has rheological properties consistent with a good dispersive viscoelastic and improved damping ability over commercial dispersive viscoelastics.

### CROSS REFERENCE TO RELATED APPLICATION This application claims the benefit of Provisional Patent Application No. 60/547,855 filed Feb. 26, 2004 and is incorporated herein by reference.

### BACKGROUND OF THE INVENTION

1. Field of the Invention This invention relates to a viscoelastic composition, method of use and related device used in viscosurgical applications and more particularly to a viscoelastic composition used in ophthalmic surgical application such as cataract removal surgery.

2. Discussion of Related Art In the past decade, advances in the technology of eye surgery have made surgical treatment of eye disease and deformities attractive to alternative therapies. Cataract removal is one of the more common surgical procedures. Cataracts are opacities of the ocular lens, which generally arise in the elderly. Typically, cataract surgery involves removal of the cataractous lens from the capsular bag and replacement of the cataractous lens with a synthetic intraocular lens. Presently, this procedure involves making an incision through the sclera or cornea into the anterior chamber of the patient's eye. Another incision is made into the capsular bag. The cataractous lens is fractured in the capsular bag by procedures such as phacoemulsification and removed from the capsular bag by procedures such as aspiration. Thereafter, an intraocular lens is inserted into the capsular bag and deployed therein. The overall procedure is potentially traumatic to the tissue surrounding the anterior chamber. It is advantageous to reduce the amount of trauma to any living tissue in the patient eye during a surgical procedure. Particularly, corneal endothelial cells in the capsular bag are sensitive to damage, which is often irreversible. Serious damage can cause loss of eyesight and failure of the surgical procedure.

Viscoelastic compositions are injected in the anterior chamber of the eye and the capsular bag during surgery to protect the tissue from physical trauma. The viscoelastic compositions provide a physical barrier or cushion between the instruments and the tissue. Furthermore, viscoelastic compositions maintain the shape of a cavity during operation including the anterior chamber and capsular bag.

In addition to cataract surgery, viscoelastic compositions are useful in reducing tissue trauma and maintaining space of a cavity during other ophthalmic surgical procedures, including but not limited to trabeculectomy and vitrectomy.

Viscoelastic materials have properties that make them effective for use in eye surgery to maintain the shape of a cavity and to protect the tissue. A viscoelastic under zero-shear or low-shear preferably has a relatively high viscosity. Higher viscosity compounds under zero-shear or low-shear conditions have better space maintenance properties than low viscosity compounds. However, it is difficult to inject or remove a highly viscous liquid through a cannula used for surgical procedures inside the eye. It is highly desirable to have a compound that has low viscosity under high-shear conditions and high viscosity under zero-shear or low-shear conditions. The ratio of the shear rate at low-shear condition to a high-shear condition is the pseudoplasticity index. It is desirable for a viscoelastic material to have high pseudoplasticity.

Common viscoelastic compositions for eye surgery include sodium hyaluronate (Healon® by Pfizer, New York, N.Y.), sodium hyaluronate and chondroitin sulfate (Viscoat® by Alcon Laboratories, Fort Worth, Tex.), hydroxypropylmethylcellulose (Ocucoat® by Bausch & Lomb, Rochester, N.Y.).

A composition whose viscoelastic component is essentially sodium hyaluronate has good shape maintaining characteristics, but is less effective at protecting the cells against damage during phacoemulsification.

A composition with hydroxypropylmethylcellulose and mixtures of hyaluronic acid and chondroitin sulfate are two viscoelastic compositions with dispersive viscoelastic properties. However, there is still a need for a dispersive viscoelastic with a relatively flat low-shear viscosity profile and improved dampening characteristics.

Alginate, for the purpose of this application is a polysaccharide that comprises β-D-mannuronic acid and α-L-guluronic acid monomers or salts or derivatives of such acids or salts.

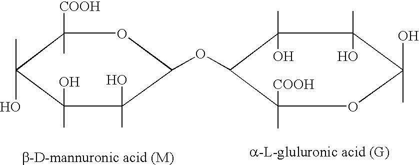

Some alginate polymers are block copolymers with blocks of the guluronic acid (or salt) monomers alternating with blocks of the mannuronic acid (or salt) monomers. Some alginate molecules have single monomers of guluronic acid (or salt) alternating with the comonomers of mannuronic acid (or salt). The ratio and distribution of the M and G components along with the average molecular weight affect the physical and chemical properties of the copolymer. See Haug, A. et al., Acta Chem Scand 20:183-190 (1966). Alginate polymers have viscoelastic rheological properties and other properties that make it suitable for some medical applications. See Klock, G. et al., Biocompatibility of manurononic acid-rich alginates, Biomaterials 18(10): 707-713 (1997).

The use of alginate as a thickener for topical ophthalmic use is disclosed in U.S. Pat. No. 6,528,465 and U.S. Publication 2003-0232089 incorporated herein by reference in their entirety. In U.S. Pat. No. 5,776,445, alginate is used as a drug delivery agent that is topically applied to the eye. Particularly, the amount of guluronic acid in the alginate was taught to exceed 50%.

While significant improvements have been made in the rheological properties of viscoelastic compositions, there still exists a need for a composition that has good adhesive properties improved dampening ability. The present invention addresses these and other needs.

### SUMMARY OF THE INVENTION

The present invention is a novel viscoelastic composition that has improved viscoelastic properties. The composition comprises an aqueous vehicle and a viscosurgically pure alginate with a minimum alginate concentration of about 0.01% w/v and a maximum alginate concentration of about 20% w/v based upon the total weight of the composition. Particularly, the viscoelastic compositions have relatively flat low-shear viscosity profile. Furthermore, the viscoelastic compositions or materials of at least one embodiment of the present invention have damping characteristics that are an improvement over dispersive viscoelastic compositions or materials for viscosurgical applications.

In another embodiment of the present invention, there is a method of temporarily maintaining space in a cavity in mammalian tissue. The method comprises injecting a viscoelastic material comprising alginate and an aqueous carrier into the cavity. At least a portion of the viscoelastic material is removed from the cavity. The alginate is viscosurgically pure.

In yet another embodiment, there is a method of protecting tissue from trauma during a surgical procedure, the method comprises coating at least a portion of the tissue with a viscoelastic material comprising an aqueous vehicle and alginate. After the tissue is coated, a surgical procedure is performed near the tissue. At least a portion of the viscoelastic material is removed from the tissue after surgical procedure is performed. In one embodiment, at least a portion of the tissue in an anterior chamber of an eye is covered during the coating step. In another application, at least a portion of the corneal endothelium of an eye is coated.

In still another embodiment, there is a package for a viscoelastic material. The package comprises a syringe containing a viscoelastic material comprising an aqueous vehicle and alginate. Optionally, the syringe has an outlet port. The package further comprises a cannula configured to sealably connect to the outlet port having a maximum inner diameter of about 1000 microns. Typically, the maximum inner diameter is about 700 microns, about 500 microns or about 300 microns.

In one embodiment, there is a method of replacing a natural lens from an eye, the method comprises the steps of:

(a) providing a passage through a sclera or cornea into an anterior chamber of the eye;

(b) removing at least a portion of the aqueous humor from the anterior chamber;

(c) inserting a viscoelastic material into the anterior chamber, the viscoelastic material comprises an aqueous vehicle and alginate;

(d) phacoemulsifying a lens in the capsular bag of the eye;

(e) removing substantially all of the lens from the capsular bag;

(f) injecting the viscoelastic material into the capsular bag; and

(g) inserting an intraocular lens into the capsular bag.

In one embodiment, there is an additional step of removing at least a portion of the viscoelastic material from the capsular bag after the intraocular lens is inserted into the capsular bag. Optionally and additionally, at least a portion of the viscoelastic material is removed from the anterior chamber. The phrase, “removing substantially all” as it relates to lenses and lens fragments, means a sufficient quantity to facilitate an effective removal of the lens. According to one embodiment, an effective removal of the lens requires removal of a minimum of about 90% w/v of the lens, about 95% w/v of the lens or about 98% w/v of the lens. Typically, the method further includes a step of suturing the sclera after the intraocular lens is inserted into the capsular bag.

In one embodiment, there is a method of inserting an intraocular lens into a capsular bag of an eye. The method comprises the steps of:

providing an eye with a cornea removed from the capsular bag and a passage through the sclera or cornea into the capsular bag;

providing a lens insertion device comprising a loadable chamber configured to receive the intraocular lens, a tapered conduit having a first end connected to the loadable chamber and a second end, the second end is configured to penetrate into the passage, and a slidable actuator configured to actuate the intraocular lens through the conduit past the second end;

coating at least a portion of the intraocular lens with a viscoelastic material comprising an aqueous vehicle and alginate;

loading the intraocular lens into the loadable chamber;

inserting the conduit into the passage;

positioning the second end inside the capsular bag;

actuating the coated intraocular lens through the conduit into the capsular bag; and

removing the conduit from the passage.

In one application, the step of coating occurs after the step of loading. Additionally and optionally, the second end of the tapered conduit has an inner diameter that is a maximum of about 5 mm. Preferably the second end of the tapered conduit has an inner diameter that is a maximum of about 4 mm about 3.5 mm, about 3 mm or about 2.8 mm.

### BRIEF DESCRIPTION OF THE DRAWINGS

FIG. 1 shows the steady state shear for an alginate formulation compared to other solutions.

FIG. 2 shows the oscillation test for an alginate formulation compared to other solutions.

### DETAILED DESCRIPTION OF THE INVENTION

The present invention is a novel viscoelastic composition that has improved viscoelastic properties. The composition comprises an aqueous vehicle and a viscosurgically pure alginate with a minimum alginate concentration of about 0.01% w/v and a maximum alginate concentration of about 20% w/v based upon the total weight of the composition. Particularly, the viscoelastic compositions or materials of at least one embodiment of the present invention is capable of maintaining a zero-shear viscosity profile at higher shear rates relative to other leading viscoelastic materials. Furthermore, the viscoelastic compositions or materials of at least one embodiment of the present invention have a damping ratio that is higher than other viscoelastic compositions or materials for viscosurgical applications.

For the purpose of this application, a viscoelastic material has relatively viscous properties under low-shear and relatively elastic properties under high-shear conditions.

Viscosurgically pure as it pertains to this application refers to a viscoelastic composition or ingredient thereof that is sufficiently pure and free of impurities to meet or exceed the United States Food and Drug Administration standards for a viscosurgical viscoelastic effective at the time this application is effective.

Alginate is a polysaccharide that comprises β-D-mannuronic acid and α-L-guluronic acid monomers or salts or derivatives of such acids or salts. Some alginate polymers are block copolymers with blocks of the guluronic acid (or salt) monomers alternating with blocks of the mannuronic acid (or salt) monomers. Some alginate molecules have single monomers of guluronic acid (or salt) alternating with the comonomers of mannuronic acid (or salt). Other variations and combinations of mannuronic acid and guluronic acid are also potentially found in the alginate polymers. Alginate is typically extracted from sources of brown seaweed including kelp. Sources for alginate include but are not limited to alginate under the trademark Pronova UP™ from FMC Biopolymers, FMC Corporation, Philadelphia, Pa. The viscoelastic composition according to one embodiment of the present invention includes an alginate that comprises β-D-mannuronic acid and α-L-guluronic acid. Particularly, the ratio of the β-D-mannuronic acid to α-L-guluronic acid is in a range having a minimum of about 1 and a maximum of about 4. Typically, the ratio of β-D-mannuronic acid to α-L-guluronic acid is in a range having a minimum of about 1.1 or about 1.2. Typically, the ratio of β-D-mannuronic acid to α-L-guluronic acid is in a range having a maximum of about 3.4, about 3, about 2 or about 1.3. Most preferably, in one embodiment, the ratio of β-D-mannuronic acid to α-L-guluronic acid is in a range having a minimum of about 1.2 and a maximum of about 1.3. Other variations and combinations of mannuronic acid and guluronic acid are also potentially found in synthetic or natural sources of alginate.

The average molecular weight of the alginate is a minimum of about 50 kD and a maximum of about 5,000 kD. Typically, the average molecular weight of the alginate is a minimum of about 50 kD, about 100 kD, about 200 kD, about 500 kD or about 1000 kD. Typically, the average molecular weight of the alginate is a maximum of about 2000 kD, about 1000 kD, about 750 kD or about 500 kD.

As noted the viscoelastic composition has a minimum alginate concentration of about 0.05% w/v and a maximum alginate concentration of about 9% w/v, based upon the total weight of the composition. Typically, the minimum alginate concentration is about 1% w/v, about 1.5% w/v, about 2% w/v, about 3% w/v or about 4% w/v based upon the total weight of the viscoelastic composition or material. Typically, the maximum alginate concentration is about 10% w/v, about 8% w/v, about 6% w/v, about 4% w/v, about 3% w/v or about 2% w/v based upon the total weight of the viscoelastic composition or material. Preferably the alginate concentration is a minimum of about 2% w/v and a maximum of about 5.25% w/v.

Preferably, in one embodiment, the average molecular weight of the alginate is a minimum of about 295 kD and a maximum of about 350 kD when the alginate concentration is a minimum of about 1% w/v and a maximum of about 3% w/v and most preferably about 2% w/v. Preferably, in one embodiment, the average molecular weight of the alginate is a minimum of about 200 kD to a maximum of about 300 kD when the alginate concentration is about 4% w/v to about 6% w/v, most preferably about 5% w/v.

Optionally, the pH is adjusted to a desired range having a minimum of about 7 and a maximum of about 8. In one embodiment, the pH of the viscoelastic composition or material is a minimum of about 7.1, about 7.2 or about 7.3 and a maximum of about 7.8, about 7.6, about 7.4 or about 7.3. The pH is adjusted with physiological acids or bases such as acetic acid, acetate, carbonic acid, carbonate, phosphoric acid, phosphate. After the pH is adjusted, the pH is typically maintained with a buffer system. Preferably, a buffer system does not substantially affect the viscoelastic properties of the viscoelastic composition or material. Desirably, the buffer system does not cause irritation at the amounts used in the viscoelastic composition or material. Buffer systems useful in the present invention include but are not limited to a N-2hydroxyethylpiperazine-N′-ethane sulphonic acid (HEPES) buffer system, a carbonate buffer system, and a phosphate buffer system—more preferably a phosphate buffered saline (PBS) system.

In one embodiment, the osmolality of the composition is a minimum of about 200 mOsmol/L and a maximum of about 400 mOsmol/L. Typically, the osmolality of the viscoelastic composition or material is a minimum of about 220 mOsmol/L, about 260 mOsmol/L, about 280 mOsmol/L, about 300 mOsmol/L or about 320 mOsmol/L. Typically, the osmolality of the viscoelastic composition or material is a maximum of about 400 mOsmol/L, about 380 mOsmol/L, about 360 mOsmol/L or about 340 mOsmol/L. Most preferably, the osmolality of the viscoelastic composition is about 340 mOsmol/L. In one embodiment, the osmolality is altered by adding an osmolality-adjusting agent known in the art. Typically, osmolality-adjusting agents are capable of increasing the osmolality of the viscoelastic composition or material without causing irritation of the eye at the quantity needed to appropriately adjust the osmolality. Suitable osmolality-adjusting agents include but are not limited to glycerin. Most preferably, the osmolality-adjusting agent is added in an amount that is a minimum of about 0.1% w/v, about 1% w/v or about 1.5% w/v and a maximum of about 5% w/v, about 2.5% w/v or about 2% w/v.

The viscoelastic properties of the viscoelastic composition of the present invention are important to their effectiveness in the surgical procedure. Zero-shear viscosity is a good indicator of how a viscoelastic material will maintain the space of a cavity in human tissue. Zero-shear viscosity is the extrapolation of the viscosity of a liquid to a zero-shear rate from measurements of viscosity as the shear rate approaches zero measured on a plate and cone rheometer at 37° C. In one embodiment, the zero-shear viscosity of the composition is a minimum of about 10 Pa-s and a maximum of about 300 Pa-s. Generally, the zero-shear viscosity of the viscoelastic composition or material is a minimum of about 50 Pa-s, about 75 Pa-s or about 100 Pa-s. Generally, the zero-shear viscosity of the viscoelastic composition or material is a maximum of about 250 Pa-s, about 200 Pa-s or about 150 Pa-s.

High-shear conditions refer to shear conditions having a minimum shear force of about 100 sec⁻¹. High-shear viscosity, for the purpose this patent application, is the viscosity of a liquid measured on a plate and cone rheometer at 37° C. with a shear rate of 1000 sec⁻¹. According to one embodiment, the high-shear viscosity of the composition is a minimum of about 0.1 Pa-s and a maximum of about 30 Pa-s. Generally, the high-shear viscosity of the viscoelastic composition or material is a minimum of about 0.5 Pa-s, about 1 Pa-s or about 2 Pa-s. Generally, the high-shear viscosity of the viscoelastic composition or material is a maximum of about 20 Pa-s, about 15 Pa-s, about 10 Pa-s, about 5 Pa-s or about 3 Pa-s.

The pseudoplasticity index is another important factor. The pseudoplasticity measures the degree of change in viscosity from a low shear state to a high shear state. For the purpose of this application, pseudoplasticity is defined as the ratio of viscosity at a shear rate of 0.3 s⁻¹ to the viscosity at a shear rate of 300 s⁻¹. According to one embodiment, the pseudoplasticity index of the viscoelastic composition is a minimum of about 80. Typically, the pseudoplasticity index of the viscoelastic composition is a minimum of about 100, about 120, about 140, about 160 or about 180. In one embodiment, the pseudoplasticity index of the viscoelastic composition is about 200.

In one embodiment, there is a method of replacing a natural lens from an eye. Examples of procedures for removing a lens from a patient's eye include but are not limited to U.S. Pat. Nos. 3,589,363 (cataract surgery), 3,693,613 (phacoemulsification) and 5,718,676 (process using micro flow needle), which are all incorporated herein by reference in their entirety. The process generally includes providing a passage through a sclera or cornea into an anterior chamber of the eye. The process involves making a small incision into the sclera or cornea. Alternatively or additionally, a cannula or trochar is used to create a passage through the sclera or cornea. Preferably, the incision or passage is as small as possible. Preferably the incision or passage is smaller than about 5 mm, about 4 mm or about 3 mm. Thereafter, the aqueous humor is withdrawn or otherwise removed from the anterior chamber of the eye.

A viscoelastic material, according to any one of the embodiments or combinations, is inserted into the anterior chamber. The viscoelastic, of one embodiment, maintains the space in the anterior chamber. The viscoelastic of one embodiment, coats the tissue in the wall of the anterior chamber.

According to one embodiment, there is a device for delivering a viscoelastic composition or material into the anterior chamber of a patient's eye. Alternatively, there is a package for viscoelastic material. The package or device comprises a syringe containing a viscoelastic material comprising an aqueous vehicle and alginate. In one embodiment, the syringe has an outlet port, the package further comprising a cannula configured to sealably connect to the outlet port having a maximum inner diameter of about 1000 microns. Typically, the maximum inner diameter is about 700 microns, about 500 microns or about 300 microns.

Once the viscoelastic material is inserted into the anterior chamber the corneal lens is removed. The technique for removing the lens includes performing a capsulorhexis incision and breaking down the lens into smaller pieces through phacoemulsification or other known techniques. Thereafter, the pieces are removed by aspiration.

The viscoelastic material is inserted into the capsular bag for space maintenance purposes. Moreover, the viscoelastic composition or material coats the capsular bag and protects it for additional steps in the surgical procedure.

According to one embodiment, the intraocular lens is inserted into the capsular bag. Typically, there is a method of inserting an intraocular lens into a capsular bag of an eye. The method comprises providing a lens insertion device comprising a loadable chamber configured to receive the intraocular lens, a tapered conduit having a first end connected to the loadable chamber and a second end. The second end is configured to penetrate through the passage in the corneal lens and into the capsular bag. An example of a lens insertion device is found in U.S. Pat. No. 6,558,419, which is incorporated herein by reference in its entirety. The lens insertion device is further configured with a slidable actuator. The slidable actuator of one embodiment is configured to actuate the intraocular lens through the conduit past the second end. Typically, the second end of the tapered conduit has an inner diameter that is a maximum of about 5 mm. Preferably the second end of the tapered conduit has an inner diameter that is a maximum of about 4 mm about 3.5 mm, about 3 mm or about 2.8 mm.

Prior to deployment, at least a portion of the intraocular lens is coated with a viscoelastic composition or material according to any one of the embodiments of the present invention. The intraocular lens is loaded into the loadable chamber either before or after it is coated. The conduit is inserted through the passage. The actuator forces the intraocular lens through the passage and into the capsular bag. After the intraocular lens is deployed, the conduit is removed from the passage.

Typically, at least a portion of the viscoelastic material is removed from the capsular bag and/or anterior chamber. A physiological solution is then used to fill the anterior chamber. The sclera and/or cornea are sutured to close the passage.

In one embodiment, of the present invention, one or more viscoelastic compositions set forth in the present invention are used to maintain the space of a cavity in a patient's tissue. The process includes injecting a viscoelastic material comprising alginate and an aqueous carrier into the cavity. After the cavity is maintained for a period of time, at least a portion of the viscoelastic material is removed from the cavity. The space is often maintained during a surgical procedure that often occurs in the cavity itself. In one embodiment, the surgery occurs in the patient's eye. In another embodiment, the surgical procedure is cataract removal. The cavity is the anterior chamber of the patient's eye and/or the capsular bag of the patient's eye.

The use of alginate in surgery also protects tissue from damage during the surgical procedure. The viscoelastic composition or material coats the surface of the tissue. A surgical procedure is performed near the tissue. The viscoelastic composition cushions the tissue from physical trauma. Preferably, the viscoelastic has dispersive viscoelastic properties to protect the tissue. In one embodiment, the process of coating covers at least a portion of the tissue in an anterior chamber of an eye. In another embodiment, the step of coating covers at least a portion of the corneal endothelium of an eye. The surgical procedure further includes removing at least a portion of the viscoelastic material from the tissue.

### EXAMPLE 1

Several viscoelastic polysaccharides were compared to determine rheological properties.

The following solutions were prepared for testing:
Solution 1: 2% Cross-linked Carboxymethylcellulose (Akucell from Akzo Nobel) with PBS buffer at pH 7.3.
Solution 2: 2% Hydroxypropylmethylcellulose with PBS buffer at pH 7.3.
Solution 3: 3% Alginate solution (satialgine, obtained from Degussa Texturants) with PBS buffer at 7.3.
Solution 4: Sodium Hyaluronate (Amvisc™ Plus, from Bausch & Lomb).
Solution 5: 3% Sodium Hyaluronate and 4% Chondroitin Sulfate (Viscoat® Alcon Labs).
   Steady Shear Test

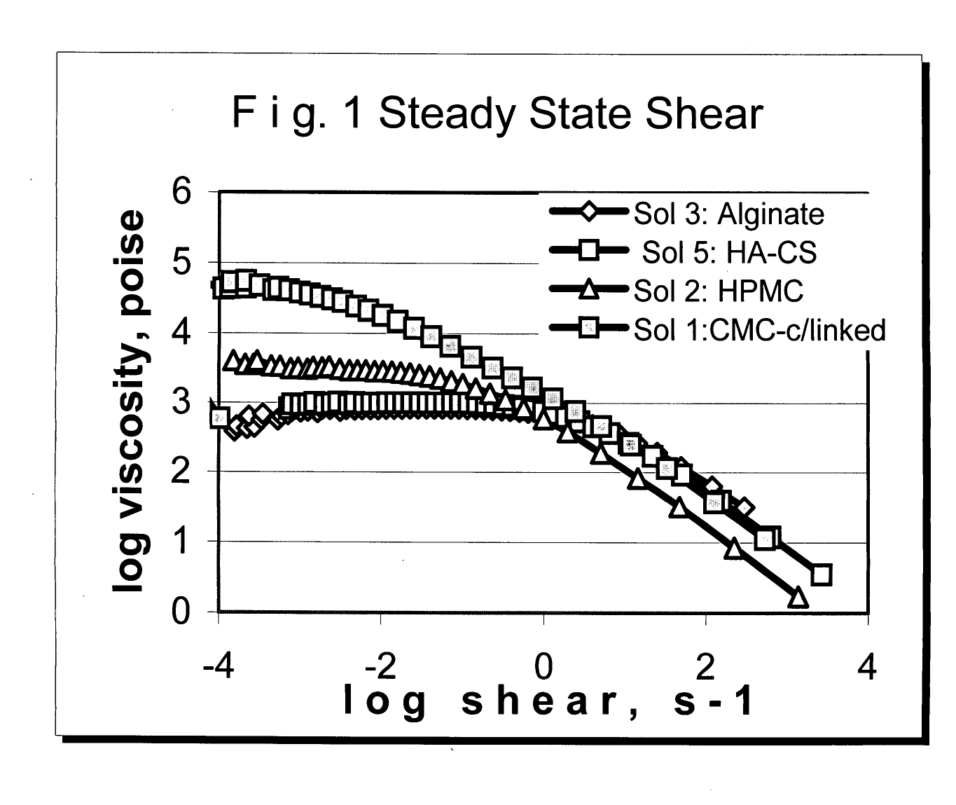

A TA Instruments T-1000 R rheometer with a 50-mm diameter cone-and-plate geometry (cone angle: 2°) was used to perform rheological tests on the above solutions under ambient conditions. The geometry gap used was 48 microns. Steady shear tests were conducted using torque as the control parameter. The results of the steady shear test are shown in FIG. 1. The steady shear test illustrated that the flat zero-shear viscosity profile, similar to Solution 5 (hyaluronic acid and chondroitin sulfate), establishes that Solution 3 (alginate) has good dispersive viscoelastic. Other viscoelastic solutions tested such as Solution 1 (carboxymethylcellulose), Solution 2 (hyroxypropylmethylcellulose), Solution 4 (hyaluronic acid) does not exhibit the flat low-shear viscosity profile necessary for a dispersive viscoelastic to protect the endothelial layer during the phacoemulsification process.

### EXAMPLE 3

### Dynamic Oscillation Test

Dynamic oscillation tests were carried out under all the conditions of Example 2, and were additionally carried out at 1% strain control for Solution 2, Solution 3 and Solution 5.

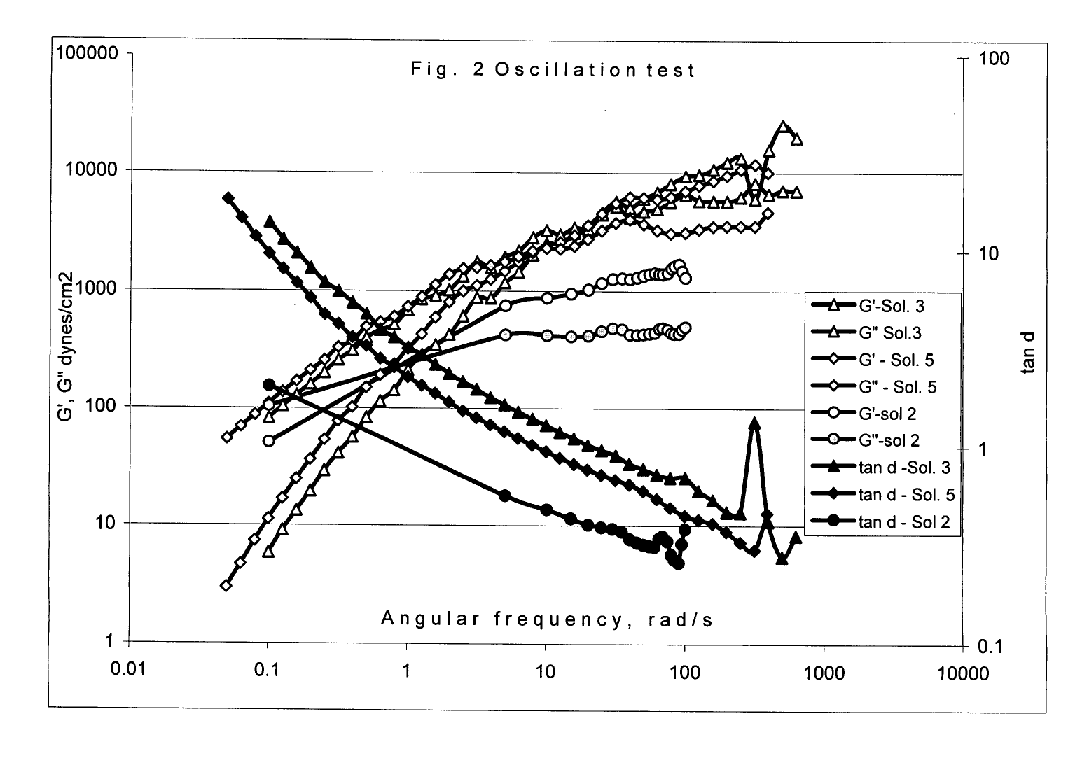

The results of the dynamic oscillation test are shown in FIG. 2, which compares the dynamic storage and loss modulus as a function of angular frequency. Solution 3 (alginate) demonstrated a similar storage and loss modulus profile as compared to Solution 5 (hyaluronic acid-chondroitin sulfate) for most of the frequency sweep. The crossover frequency for G′ and G″ are similar for the two viscoelastics. A comparison of the damping ratio, tanδ (=G″/G′) profiles indicates a better damping or input stress dissipation ability of Solution 3 relative to Solution 5. 0 f particular interest is the damping region around the peak at 315 rad/s for Solution 3, which indicates that Solution 3 will dissipate the phaco-energy more effectively than Solution 5, especially at high ultrasonic frequencies, which will in turn prevent damage to the endothelial cells.

1. A composition comprising an aqueous vehicle and a viscosurgically pure alginate with a minimum alginate concentration of about 0.01% w/v and a maximum alginate concentration of about 20% w/v based upon the total weight of the composition.

2. The composition of claim 1, wherein the average molecular weight of the alginate is a minimum of about 50 kD and a maximum of about 5,000 kD.

3. The composition of claim 1, wherein the composition has a minimum alginate concentration of about 0.05% w/v and a maximum alginate concentration of about 9% w/v, based upon the total weight of the composition.

4. The composition of claim 1, wherein the osmolality of the composition is a minimum of about 200 mOsmol/L and a maximum of about 400 mOsmol/L.

5. The composition of claim 1, wherein the zero-shear viscosity of the composition is a minimum of about 10 Pa-s and a maximum of about 300 Pa-s.

6. The composition of claim 1, wherein the high-shear viscosity of the composition is a minimum of about 0.1 Pa-s and a maximum of about 30 Pa-s.

7. The composition of claim 1, wherein the pseudoplasticity index of the viscoelastic composition is a minimum of about 100.

8. The composition of claim 1, wherein the alginate comprises β-D-mannuronic acid and α-L-guluronic acid, wherein the ratio of the β-D-mannuronic acid to α-L-guluronic acid is in a range having a minimum of about 1 and a maximum of about 4.

9. The composition of claim 1, wherein the pH of the composition is a minimum of about 7 and a maximum of about 8.

10. A method of temporarily maintaining space in a cavity in mammalian tissue, the method comprising the steps of:
(a) injecting a viscoelastic material comprising alginate and an aqueous carrier into the cavity; and
(b) removing at least a portion of the viscoelastic material from the cavity.

11. The method of claim 10, wherein the alginate is a viscosurgically pure alginate.

12. The method of claim 11, wherein the alginate concentration is a minimum of about 0.01% w/v and a maximum of about 20% w/v based upon the total weight of the viscoelastic material.

13. The method of claim 11, wherein the average molecular weight of the alginate is a minimum of about 50 kD and a maximum of about 5,000 kD.

14. The method of claim 11, wherein the viscoelastic material has a minimum alginate concentration of about 0.05% w/v and a maximum alginate concentration of about 9% w/v, based upon the total weight of the viscoelastic material.

15. The method of claim 10, wherein the osmolality of the viscoelastic material is a minimum of about 200 mOsmol/L and a maximum of about 400 mOsmol/L.

16. The method of claim 10, wherein the zero-shear viscosity of the viscoelastic material is a minimum of about 10 Pa-s and a maximum of about 300 Pa-s.

17. The method of claim 10, wherein the high-shear viscosity of the viscoelastic material is a minimum of about 0.1 Pa-s and a maximum of about 30 Pa-s.

18. The method of claim 10, wherein the viscoelastic material has a pseudoplasticity index that is a minimum of about 100.

19. The method of claim 10, wherein the alginate comprises wherein the alginate comprises β-D-mannuronic acid and α-L-guluronic acid, wherein the ratio of the βP-D-mannuronic acid to α-L-guluronic acid is in a range having a minimum of about 1 and a maximum of about 4.

20. The method of claim 10, wherein the pH of the viscoelastic material is a minimum of about 7 and a maximum of about 8.

21. A method of protecting tissue from trauma during a surgical procedure, the method comprising the steps of:
(a) coating at least a portion of the tissue with a viscoelastic material comprising an aqueous vehicle and alginate;
(b) performing a surgical procedure near the tissue after the step (a) coating; and
(c) removing at least a portion of the viscoelastic material from the tissue before the step (b) performing.

22. The method of claim 21, wherein the step (a) coating covers at least a portion of the tissue in an anterior chamber of an eye.

23. The method of claim 21, wherein the step (a) coating covers at least a portion of the corneal endothelium of an eye.

24. The method of claim 21, wherein the alginate is a viscosurgically pure alginate.

25. The method of claim 21, wherein the alginate has a concentration that is a minimum of about 0.01% w/v and a maximum of about 20% w/v based upon the total weight of the viscoelastic material.

26. The method of claim 21, wherein the average molecular weight of the alginate is a minimum of about 50 kD and a maximum of about 5,000 kD.

27. The method of claim 21, wherein the viscoelastic material has a minimum alginate concentration of about 0.05% w/v and a maximum alginate concentration of about 9% w/v, based upon the total weight of the viscoelastic material.

28. The method of claim 21, wherein the osmolality of the viscoelastic material is a minimum of about 200 mOsmol/L and a maximum of about 400 mOsmol/L.

29. The method of claim 21, wherein the zero-shear viscosity of the viscoelastic material is a minimum of about 10 Pa-s and a maximum of about 300 Pa-s.

30. The method of claim 21, wherein the high-shear viscosity of the viscoelastic material is a minimum of about 0.1 Pa-s and a maximum of about 30 Pa-s.

31. The method of claim 21, wherein the alginate comprises β-D-mannuronic acid and α-L-guluronic acid, wherein the ratio of the β-D-mannuronic acid to α-L-guluronic acid is in a range having a minimum of about 1 and a maximum of about 4.

32. The method of claim 21, wherein the pH of the viscoelastic material is a minimum of about 7 and a maximum of about 8.

33. A package for a viscoelastic material, the package comprising a syringe containing a viscoelastic material comprising an aqueous vehicle and alginate.

34. The package of claim 33, wherein the syringe has an outlet port, the package further comprising a cannula configured to sealably connect to the outlet port having a maximum inner diameter of about 1000 microns.

35. The package of claim 33, wherein the average molecular weight of the alginate is a minimum of about 50 kD and a maximum of about 5,000 kD.

36. The package of claim 33, wherein the viscoelastic material has a minimum alginate concentration of about 0.05% w/v and a maximum alginate concentration of about 9% w/v, based upon the total weight of the viscoelastic material.

37. The package of claim 33, wherein the osmolality of the viscoelastic material is a minimum of about 200 mOsmol/L and a maximum of about 400 mOsmol/L.

38. The package of claim 33, wherein the zero-shear viscosity of the viscoelastic material is a minimum of about 10 Pa-s and a maximum of about 300 Pa-s.

39. The package of claim 33, wherein the high-shear viscosity of the viscoelastic material is a minimum of about 0.1 Pa-s and a maximum of about 30 Pa-s.

40. The method of claim 33, wherein the viscoelastic material has a pseudoplasticity index that is a minimum of about 100.

41. The package of claim 33, wherein the alginate comprises β-D-mannuronic acid and α-L-guluronic acid, wherein the ratio of the β-D-mannuronic acid to α-L-guluronic acid is in a range having a minimum of about 1 and a maximum of about 4.

42. The package of claim 33, wherein the pH of the viscoelastic material is a minimum of about 7 and a maximum of about 8.

43. A method of replacing a natural lens from an eye, the method comprising the steps of:
(a) providing a passage through a sclera or cornea into an anterior chamber of the eye;
(b) removing at least a portion of the aqueous humor from the anterior chamber;
(c) inserting a viscoelastic material into the anterior chamber, the viscoelastic material comprises an aqueous vehicle and alginate;
(d) phacoemulsifying a lens in the capsular bag of the eye;
(e) removing substantially all of the lens from the capsular bag;
(f) injecting the viscoelastic material into the capsular bag; and
(g) inserting an intraocular lens into the capsular bag.

44. The method of claim 43, further comprising the step of removing at least a portion of the viscoelastic material from the capsular bag.

45. The method of claim 43, further comprising the step of removing at least a portion of the viscoelastic material from the anterior chamber.

46. The method of claim 43, further comprising the step of suturing the sclera after the step (g) inserting an intraocular lens.

47. The method of claim 43, wherein the alginate is a viscosurgically pure alginate.

48. The method of claim 43, wherein the alginate concentration is a minimum of about 0.01% w/v and a maximum of about 20% w/v based upon the total weight of the viscoelastic material.

49. The method of claim 43, wherein the average molecular weight of the alginate is a minimum of about 50 kD and a maximum of about 5,000 kD.

50. The method of claim 43, wherein step (d) removing further comprises removing the lens by a procedure selected from the group consisting of extracapsular cataract extraction and phacoemulsification.

51. The method of claim 43, wherein the osmolality of the viscoelastic material is a minimum of about 200 mOsmol/L and a maximum of about 400 mOsmol/L.

52. The method of claim 43, wherein the zero-shear viscosity of the viscoelastic material is a minimum of about 10 Pa-s and a maximum of about 300 Pa-s.

53. The method of claim 43, wherein the high-shear viscosity of the viscoelastic material is a minimum of about 0.1 Pa-s and a maximum of about 30 Pa-s.

54. The method of claim 43, wherein the viscoelastic material has a pseudoplasticity index that is a minimum of about 100.

55. The method of claim 43, wherein the alginate comprises β-D-mannuronic acid and α-L-guluronic acid, wherein the ratio of the β-D-mannuronic acid to α-L-guluronic acid is in a range having a minimum of about 1 and a maximum of about 4.

56. The method of claim 1, wherein the pH of the viscoelastic material is a minimum of about 7 and a maximum of about 8.

57. A method of inserting an intraocular lens into a capsular bag of an eye, the method comprising the steps of:
providing an eye with a cornea removed from the capsular bag and a passage through the sclera or cornea into the capsular bag;
providing a lens insertion device comprising a loadable chamber configured to receive the intraocular lens, a tapered conduit having a first end connected to the loadable chamber and a second end, the second end is configured to penetrate into the passage, and a slidable actuator configured to actuate the intraocular lens through the conduit past the second end;
coating at least a portion of the intraocular lens with a viscoelastic material comprising an aqueous vehicle and alginate;
loading the intraocular lens into the loadable chamber;
inserting the conduit into the passage;
positioning the second end inside the capsular bag;
actuating the coated intraocular lens through the conduit into the capsular bag; and
removing the conduit from the passage.

58. The method of claim 57, wherein the step of coating occurs after the step of loading.

59. The method of claim 57, wherein the second end of the tapered conduit has an inner diameter that is a maximum of about 5 mm.

60. The method of claim 57, wherein the alginate is a viscosurgically pure alginate.

61. The method of claim 57, wherein the alginate concentration is a minimum of about 0.01% w/v and a maximum of about 20% w/v based upon the total weight of the viscoelastic material.

62. The method of claim 57, wherein the zero-shear viscosity of the viscoelastic material is a minimum of about 10 Pa-s and a maximum of about 300 Pa-s.

63. The method of claim 57, wherein the high-shear viscosity of the viscoelastic material is a minimum of about 0.1 Pa-s and a maximum of about 30 Pa-s.
